(19) United States
(12) Patent Application Publication
(10) Pub. No.: US 2006/0241671 A1
(43) Pub. Date: Oct. 26, 2006

(54) Self-sealing surgical access port

(75) Inventor: E. Skott Greenhalgh, Wyndmoor, PA (US)

Correspondence Address: SYNNESTVEDT & LECHNER, LLP, 2600 ARAMARK TOWER, 1101 MARKET STREET, PHILADELPHIA, PA, 191072950, US

(73) Assignee: Stout Medical Group, LLC, Perkasie, PA (US)

(21) Appl. No.: 11/407,987

(22) Filed: Apr. 20, 2006

Related U.S. Application Data
(60) Provisional application No. 60/673,072, filed on Apr. 20, 2005.
(60) Provisional application No. 60/714,284, filed on Sep. 6, 2005.

(51) Int. Cl. A61M 29/00

(52) U.S. Cl. 606/191

(57) ABSTRACT

An access port for performing surgical procedures within an insufflated body cavity is disclosed. The port is formed from a duct that is inserted into the body cavity, the duct having a stiff sidewall to prevent collapse. The duct contains a flexible tube that has a constricted shape that forms a seal around a surgical instrument inserted through the duct. The tube has an inner membrane with a low coefficient of friction to facilitate passage of surgical instruments through the duct. The tube has an elastic outer membrane to bias it into the constricted shape. The tube may also be pressurized into the constricted shape by gas, gel or liquid. The tube itself may also form a seal in the absence of the tool, or a second seal, such as a duckbill valve, may be positioned within the duct.

### CROSS-REFERENCE TO RELATED APPLICATIONS

This application is based on and claims priority to U.S. Provisional Application No. 60/673,072, filed Apr. 20, 2005, and U.S. Provisional Application No. 60/714,284, filed Sep. 6, 2005.

### FIELD OF THE INVENTION

The invention concerns self sealing access ports, and especially ports usable to provide access to body cavities for surgical procedures.

### BACKGROUND OF THE INVENTION

Laparoscopy and laparoscopic surgical techniques allow various abdominal organs such as the liver, gallbladder, spleen, peritoneum, diaphragm, as well as portions of the colon and small bowel to be readily visualized and operated upon. For example, lesions on an organ may be biopsied, an organ may be sectioned, and contrast material may be injected into the organ to assist in the visualization of vascular as well as other systems.

During such procedures, the abdominal cavity is inflated with a gas such as air or nitrous oxide to create a working space in which laparoscopic surgical tools and cameras may be deployed to effect examination and various surgical procedures. Such tools may include, for example, scissors, scalpels, clamps, syringes and electro-coagulation devices to control bleeding.

It is clear from the above description that if surgical tools are to be inserted, manipulated and withdrawn from the outside of an abdominal cavity that is expanded using internal pressure, there must be a port which provides access to the cavity while also maintaining the inflation pressure within the cavity during insertion, manipulation and removal of the tools during the surgical procedure.

In addition to providing access to the abdominal cavity while maintaining a substantially fluid tight seal during the insertion, removal and manipulation of surgical tools, the access port should also have acceptable characteristics for snag resistance and push through and removal force. Snag resistance refers to the propensity of surgical tools to catch or snag on the surface of a seal, and not slide smoothly over it. If the seal surface is prone to snagging the tool, it can lead to seal damage, such as tears that compromise the fluid tightness of the seal. Push through and removal forces refer to the manual force necessary to insert or remove a tool through the access port. Excessive insertion or removal force, which can be caused by snagging or by too tight a contact force between engaging surfaces effecting the fluid tight seal, is to be avoided as it also may lead to seal damage, patient injury, as well as increase the overall difficulty of performing the procedure. There is clearly a need for a surgical access port that provides a substantially fluid tight seal while maintaining adequate snag resistance characteristics and acceptable push through and removal force requirements.

### SUMMARY OF THE INVENTION

The invention concerns a self-sealing surgical access port permitting a surgical tool to be used within a body cavity, for example, during laparoscopic surgery. The access port comprises a rigid duct having a distal end positionable within the body cavity. The duct is extendable through living tissue of the body and has a proximal end positionable outside of the cavity. A flexible tube is positioned substantially coaxially within the duct and is attached thereto. The tube has an inner low-friction surface surrounded by an elastic membrane. The membrane is biased so as to form a constricted region of the tube. The tube is elastically deformable radially outwardly to permit the surgical tool to pass through the duct and into the body cavity. The constricted region of the membrane closes around the tool to substantially continuously seal the tube while the tool extends therethrough. Preferably, the tube does not form a seal in the absence of a tool extending through the duct. The duct is sealed by a second seal in this embodiment. Alternately, the inner surface of the tube may be in contact with itself at the constricted region so as to form a seal in the absence of a tool extending through the duct.

Preferably, the tube comprises a laminate formed from a low-friction membrane forming the inner surface, the low-friction membrane being surrounded by the elastic membrane. In a preferred embodiment, the low-friction membrane comprises expanded polytetrafluoroethylene. The elastic membrane is formed from material selected from the group consisting of rubber, polyurethane and silicone. The low-friction membrane may be substantially continuously attached to the elastic membrane, or the two membranes may be merely in contact with one another.

The tube may have one of many different shapes, such as a half or a symmetrical conical shape or an hourglass shape. The tube may also have a plurality of slits extending through the elastic membrane adjacent to the constricted region, the slits augmenting the flexibility of the elastic membrane. Reinforcement of the tube is feasible using a plurality of filamentary members extending lengthwise along it. To further control the biasing, the elastic membrane may be thinner or thicker in the constricted region than in regions adjacent to the constricted region. The tube may also have a plurality of corrugations therein, the corrugations also increasing the flexibility of the tube.

In an alternate embodiment, the access port according to the invention comprises a rigid duct having a distal end positionable within the body cavity, the duct being extendable through living tissue of the body and having a proximal end positionable outside of the cavity. A flexible tube is positioned substantially coaxially within the duct and attached thereto. The tube has an inner low-friction surface surrounded by an elastic membrane. The tube also has opposite ends attached to the duct so as to define a pocket between the tube and the duct, the pocket being positioned between the opposite ends of the tube. The pocket is pressurized with a fluid so that a constricted region is formed between the ends of the tube wherein the inner surface is in contact with itself around the tube so as to form a seal closing the duct. The tube is elastically deformable radially outwardly against the pressurization to permit the surgical tool to pass through the duct and into the body cavity. The pressurization forces the constricted region of the membrane to close around the tool to substantially continuously seal the tube while the tool extends therethrough. Preferably, the pressurization forces do not seal the tube in the absence of a tool extending through the duct. Sealing of the duct in this configuration is effected by a separate second seal. Preferably, the flexible tube is shaped so as to occupy the central portion of the duct. Pressure relief is provided to allow gas or fluid between the tube and the duct to vent or escape when a surgical tool is inserted through the duct. Venting may be provided in the form of slits through the distal portion of the flexible tube or through openings in the duct side wall.

As with the first embodiment, the second embodiment also preferably comprises a laminate formed from a low-friction membrane forming the inner surface, the low-friction membrane being surrounded by the elastic membrane.

The fluid pressurizing the pocket may be a gas, a liquid or a gel. Furthermore, the elastic membrane may also be biased into a constricted shape so as to augment the biasing of the constricted region of the tube to ensure a tight seal around the tool inserted through the duct.

### BRIEF DESCRIPTION OF THE DRAWINGS

FIG. 1 is a longitudinal sectional view of an embodiment of a surgical access port according to the invention;

FIG. 1A is a longitudinal sectional view of the surgical access port shown in FIG. 1;

FIG. 2 is a cross-sectional view taken at line 2-2 of FIG. 1;

FIGS. 3-7 and 8-11 are longitudinal sectional views of various embodiments of the surgical access port according to the invention;

FIG. 7A is an exploded view of a tube embodiment used with a surgical access port according to the invention; and

FIG. 12 is a longitudinal sectional view of another embodiment of a surgical access port according to the invention.

### DETAILED DESCRIPTION OF THE EMBODIMENTS

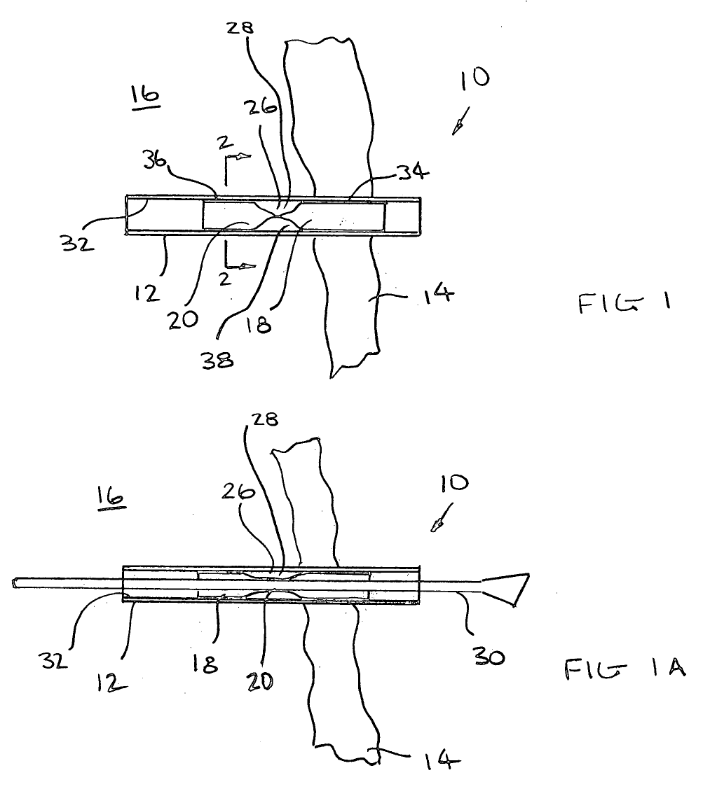

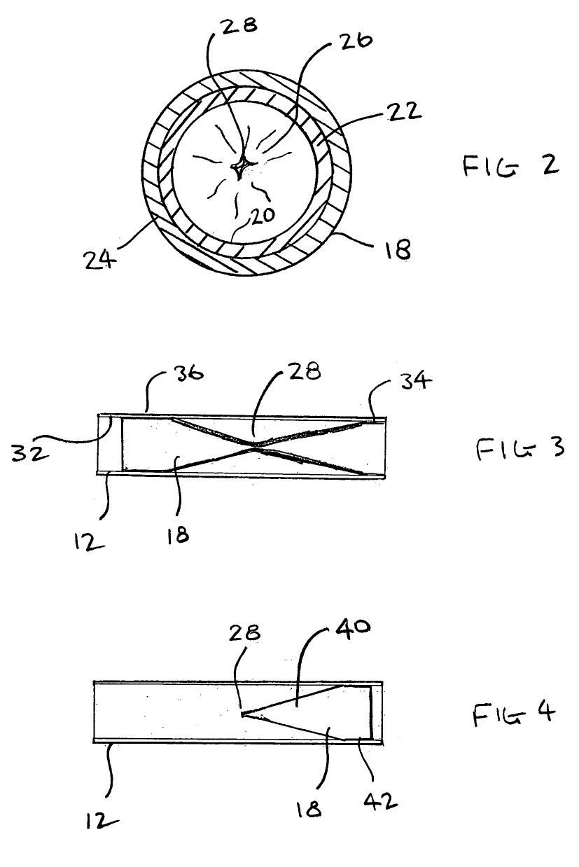

FIG. 1 shows a longitudinal sectional view of a self sealing surgical access port 10 according to the invention. Access port 10 comprises a substantially rigid duct 12 that is inserted through living tissue 14 into a body cavity 16 wherein the surgical procedure is to be performed. Cavity 16 is pressurized with a gas such as air or nitrous oxide to expand the cavity and provide room therein to insert and manipulate surgical tools for the procedure. Pressurization is effected by another access port, not shown. The tissue 14 is forced to seal against the duct 12 by inserting the duct through an opening having a smaller inner diameter than the outer diameter of the duct, thus using the inherent flexibility, resilience and elasticity of the tissue to maintain a seal against the internal pressure within the cavity 16.

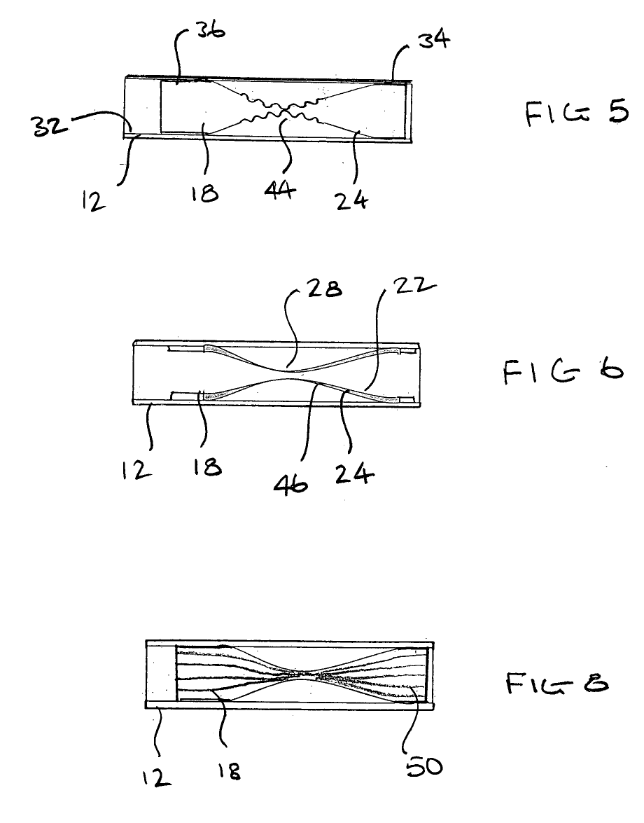

Duct 12 is preferably cylindrical in shape and may be constructed of nylon as well as other biocompatible materials such as PET, PP, PTFE and polypropylene. A flexible tube 18 is positioned substantially coaxially within the duct. As best shown in cross section in FIG. 2, flexible tube 18 has an inner surface 20 that has a low coefficient of friction to permit surgical tools to slide easily over the surface and reduce the propensity of the tools to snag when they are inserted or removed through the duct 12. Preferably, the low-friction inner surface is provided by a tubular inner membrane 22 formed from a low-friction material such as expanded polytetrafluoroethylene. An elastic outer membrane 24 surrounds the low-friction inner membrane 22 and is biased so as to form a constricted region 26 wherein the inner surface 20 is in contact with itself around the inner membrane 22 so as to form a seal 28 closing the duct. Because the outer membrane is elastic, the flexible tube 18 is elastically deformable radially outwardly to permit surgical tools such as 30 to pass through the duct and into the cavity, as shown in FIG. 1A. The biasing of the outer membrane forces the inner surface 20 against the tool 30 and thus continues to provide a fluid seal of the duct 12 even when the tool is inserted through the duct, manipulated within cavity 16 and withdrawn from the duct.

The outer membrane 24 is preferably formed from an elastomeric material, such as rubber, urethane or silicone which permits the tube 18 to have a strong elastic bias to effect the seal, and yet be repeatably elastically deformed to accommodate tools passing through the tube and still effect a fluid tight seal, either in the presence or absence of the tools. Biasing of the elastic membrane into a particular shape may be conveniently effected by molding, for example. Preferably, the inner and outer membranes 22 and 24 are substantially continuously attached to one another over their surfaces, but they may also be attached to one another at discrete points, for example, at each end of the tube 18.

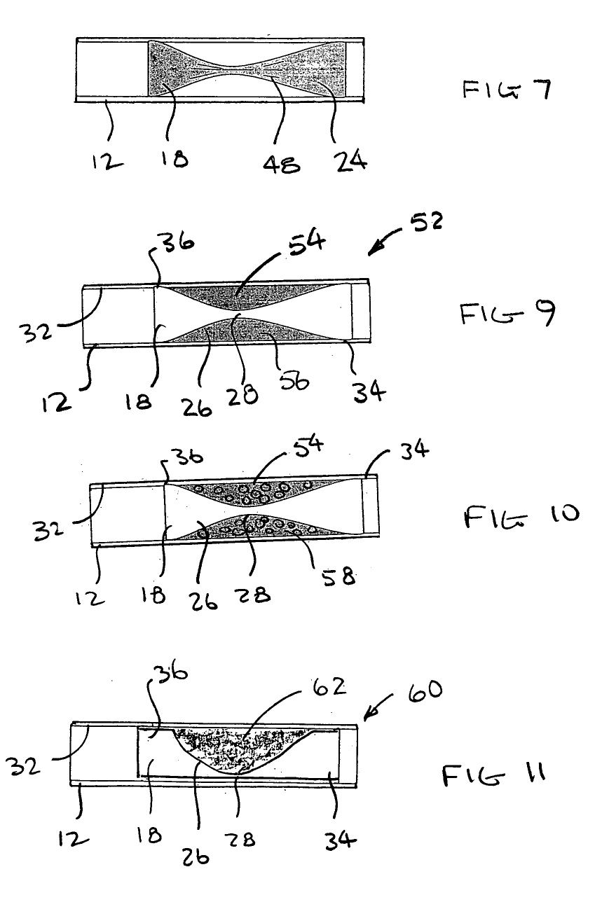

Tube 18 is attached to the inside surface 32 of duct 12 in different ways depending upon the particular design of the tube. For example, in FIG. 1, tube 18 has an hourglass shape, and is attached to inside surface 32 of duct 12 at end regions 34 and 36, the middle region 38, which must be free to expand and contract to accommodate tools and effect a seal 28, is not attached to the duct 12. Similarly, as shown in FIG. 3, when the tube 18 is molded into a double cone shape, there are again end regions 34 and 36 attached to the duct 12. The end regions are attached substantially continuously around the inside surface 32 to maintain fluid integrity of the access port. FIG. 4 shows a tube 18 in the form of a half cone 40, the half cone having a circumferential attachment region 42 at one end. Tube 18 may be adhesively bonded to the duct 12, or the components may be fused to one another if the materials are compatible.

To create a fluid tight seal 28, the elastic forces biasing the membranes 22 and 24 to form the constricted region 26 must be sufficiently high so as to maintain the constriction against inflation pressure within cavity 16. Furthermore, the biasing must also squeeze and seal against any tools that extend through the duct 12. Additionally, the membranes 22 and 24 must be sufficiently compliant so that, at the seal 28, they accommodate themselves to the cross sectional shape of any tool used with the duct to provide a fluid tight seal with one or more tools extending through the duct.

Although the need for a fluid tight seal would appear to favor membrane designs having high biasing force, a competing interest mitigating against high biasing force is the force necessary to push or pull a tool through the seal 28 within duct 12. These so called push through and removal forces determine the amount of manual force that a surgeon must exert to position a tool within the cavity, manipulate it and remove it therefrom. The higher the biasing force creating the seal, the higher the push through and removal force. High push through and removal forces are undesirable because they inhibit precise use of the tools, are fatiguing to the surgeon and carry an increased risk of snagging the tool and tearing the membranes creating the seal, as well as potential injury to the patient. Although the low-friction inner surface 20 helps guard against these disadvantages, it is also advantageous to balance the membrane biasing force so that a fluid tight seal is maintained while achieving acceptable push through and removal force facilitating manual manipulation of surgical tools within the port 10. Elastic materials capable of large elongation and having a low elastic modulus (on the order of 100-1000 psi) are feasible for forming the elastic membrane 24.

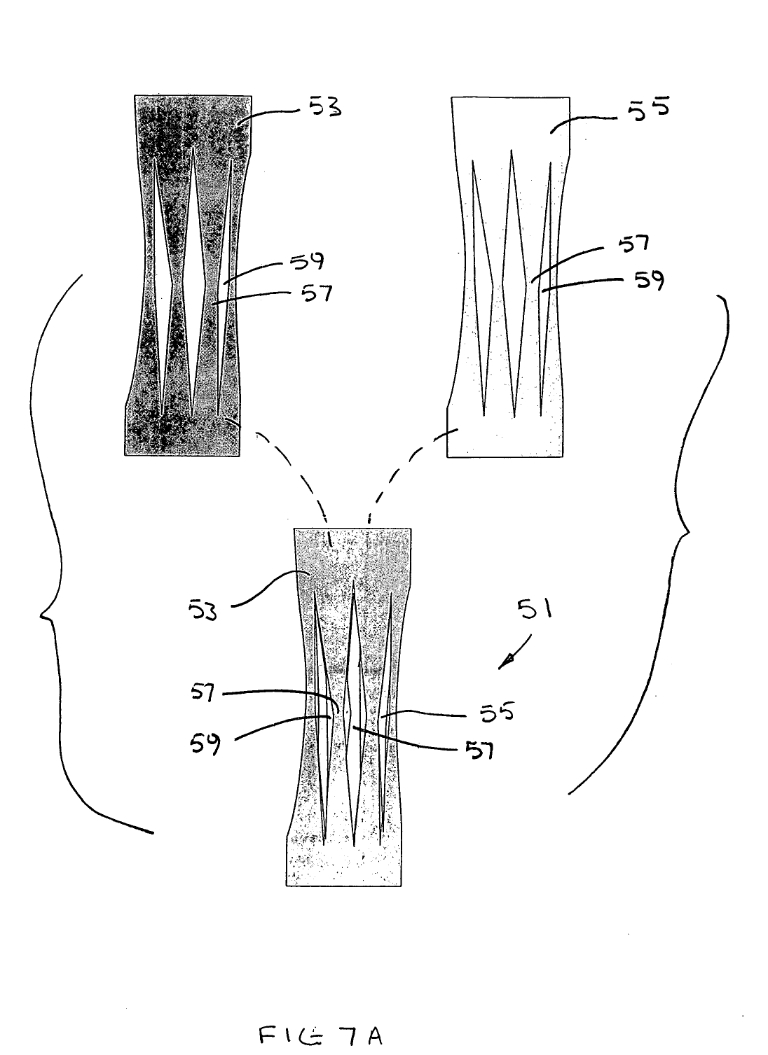

The biasing characteristics of the elastic membrane 24 may also be controlled by the shape of the membrane. For example, as shown in FIG. 5, corrugations 44 may be added at strategic points of the membrane 24 to increase the flexibility and reduce the push through and removal forces. Biasing force may also be tuned by varying the thickness of the membrane 24. This embodiment is shown in FIG. 6, wherein the tube 18 is formed from an elastic membrane 24 surrounding a low-friction membrane 22, the elastic membrane 24 having a sidewall 46 that varies in thickness, being thinner near the point of maximum constriction where seal 28 is formed to reduce push through and removal force. Further stiffness tuning may be effected as shown in FIG. 7 by positioning slits 48 in the elastic membrane 24, the slits increasing the radial flexibility of the tube 18.

Another factor that affects the behavior of the seal 28 is the hardness of the materials forming the membranes 22 and 24. Soft, compliant materials provide excellent sealing characteristics and readily accommodate irregular shapes of tools, thereby ensuring that a fluid tight seal is maintained during insertion and manipulation of the tools. Softer materials also exhibit the ability to use the internal pressure within the cavity 16 to enhance the sealing force. However, soft materials are also prone to snagging tools and excessive elongation in response applied force, increasing the potential for tears. Again, the softness of the material must be balanced so that adequate sealing characteristics are achieved without the disadvantages of excessive snagging and elongation. Materials having durometers on the order of 20 Shore A scale are feasible.

For embodiments of the invention wherein the tube need not seal in the absence of a tool extending through the duct, the tube may have an outer shell formed from a relatively stiff plastic such as nylon, PET, ABS and acrylic (Plexiglas) and an inner membrane of expanded polytetrafluoroethylene to reduce friction. FIG. 7A shows a tube 51 having a stiff outer shell 53 and an inner membrane 55. The shell and membrane are formed by expanding slotted tubes to create struts 57 with slots 59 between them and then aligning the slots with the struts when the membrane is inserted within the stiffer shell. The stiffness of the tube is determined largely by the area moment of inertia of the struts and the elastic modulus of the material comprising the shell 53 forming the tube.

As shown in FIG. 8, excessive elongation may be controlled by embedding filamentary members 50 lengthwise in either or both of the membranes 22 and 24. The filamentary members preferably have a higher elastic modulus than the membranes and act as reinforcements to limit elongation and mitigate the potential for the membranes to tear when subjected to tensile forces, for example, as encountered during insertion or removal of tools.

FIG. 9 illustrates another embodiment 52 of a surgical access port wherein biasing of the tube 18 to form a constricted region 26 with a seal 28 is accomplished by forming a pocket 54 between the ends 34 and 36 of tube 18 that are attached to the duct 12. Pocket 54 is then pressurized with a medium 56, for example, a gas, a liquid or a gel, and assumes a constricted shape depending upon the shape of the pocket 54. The pocket may also be filled with a resilient, elastic foam 58 as shown in FIG. 10. In the aforementioned examples, pocket 54 is annular and results in the tube 18 being forced into an hourglass shape. FIG. 11 shows another example embodiment 60, wherein the pocket 62 is hemi-cylindrical in the region where the seal 28 is formed. The hemi-cylindrical pocket is formed by attaching half of the tube 18 to the duct 12 over the tube's length, and attaching the end regions 34 and 36 over their entire circumference. This permits the pressure within the pocket to collapse the tube 18 onto itself from one side to form the seal 28.

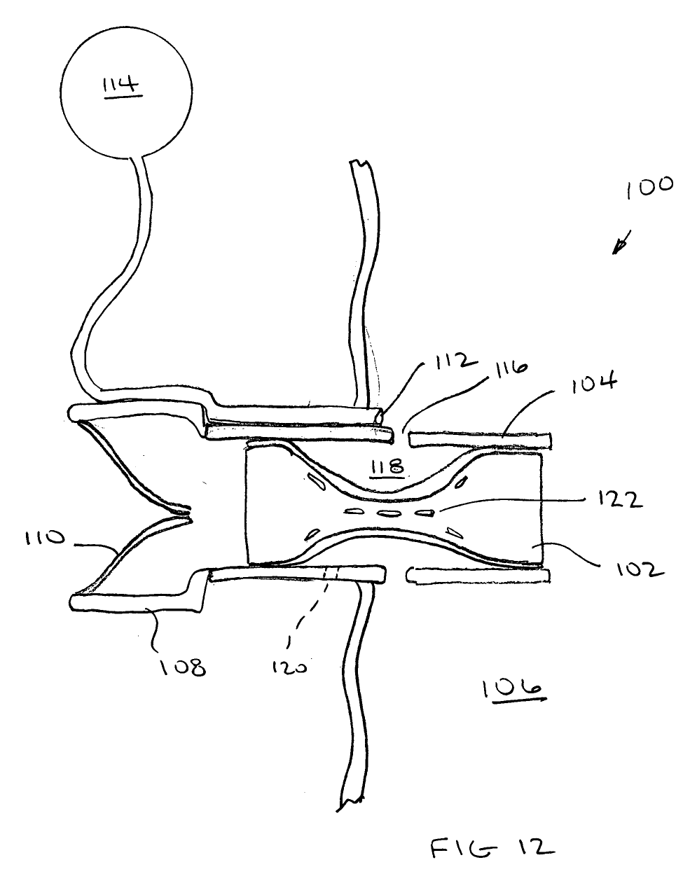

In another embodiment of a surgical access port 100 shown in FIG. 12, a flexible tube 102 is mounted within a duct 104. The proximal end of the duct (disposed outside a body cavity 106) is connected to a housing 108. A duckbill or zero seal valve 110 is mounted within the housing 108 to form a seal when no instrument is inserted into the access port 100. An insufflation port 112 also is provided to permit the body cavity 106 to be inflated to perform laparoscopic surgery. The port 112 is in fluid communication with the body cavity 106 and a source of pressurized fluid 114. One or more apertures 116 are formed in the duct 104 and act as vent holes communicating with the space 118 between the duct and the flexible tube 102. As an instrument is inserted through the access port 100 the flexible tube 102 is deformed radially outwardly, and gas or liquid in the space 118 is vented into the body cavity 106. Similarly, when the instrument is removed and the flexible tube elastically returns to its biased constricted shape occupying a portion of the interior of the duct, gas or liquid from the insufflated body cavity 106 is permitted to enter the space 118 between the flexible tube 102 and the duct 104. To further advantage, pressure from the insufflated body cavity may enhance the return of the flexible tube to the desired shape after the instrument is removed. It is also contemplated that the space between the flexible tube and duct may be vented outside the body rather than into the body cavity by ports 120, shown in phantom line. In a further alternate embodiment, tube 102 may have slits or openings 122 to allow venting of the space between the flexible tube and duct. However, care must be taken so that such slits or openings do not catch or snag on instruments as they are inserted and removed from the duct.

In the embodiment shown in FIG. 12, the flexible tube 102 is not relied upon to seal the surgical access port when no instrument is present. Rather, as noted above, the duckbill valve 110 forms a seal of the access port when no instrument is inserted. When an instrument is inserted into the access port, a seal is formed between the flexible tube 102 and the instrument. Flexible tube 102 is preferably configured to form a seal with instruments having shaft diameters ranging from about 2 mm to about 20 mm or more and preferably about 5 mm to about 12 mm.

Access ports according to the invention facilitate the use of surgical tools in laparoscopic surgical procedures by maintaining an adequate seal while avoiding the disadvantages of snagging and excessive push through and removal forces that might otherwise limit the usefulness of laparoscopic surgical techniques.

What is claimed is:

1. A self sealing surgical access port permitting a surgical tool to be used within a body cavity, said access port comprising:
a rigid duct having a distal end positionable within the body cavity, said duct being extendable through living tissue of said body and having a proximal end positionable outside of said cavity; and
a flexible tube positioned substantially coaxially within said duct and attached thereto, said tube having an inner low-friction surface surrounded by an elastic membrane, said membrane being biased so as to form a constricted region of said tube, said tube being elastically deformable radially outwardly to permit said surgical tool to pass through said duct and into said body cavity, said constricted region of said membrane closing around said tool to substantially continuously seal said tube while said tool extends therethrough.

2. A surgical access port according to claim 1, wherein said inner surface is in contact with itself around said tube so as to form a seal closing said duct.

3. A surgical access port according to claim 1, further comprising a valve positioned in said duct for sealing said duct in the absence of a surgical tool inserted therethrough.

4. A surgical access port according to claim 3, wherein said valve comprises a duckbill valve.

5. A surgical access port according to claim 1, wherein said tube comprises a laminate formed from a low-friction membrane forming said inner surface, the low-friction membrane being surrounded by said elastic membrane.

6. A surgical access port according to claim 1, wherein said low-friction membrane is substantially continuously attached to said elastic membrane.

7. A surgical access port according to claim 1, wherein said tube has a conical shape.

8. A surgical access port according to claim 1, wherein said tube has an hourglass shape, said tube having opposite ends attached to said duct.

9. A surgical access port according to claim 1, further comprising a plurality of slits extending through said elastic membrane adjacent to said constricted region, said slits augmenting the flexibility of said elastic membrane.

10. A surgical access port according to claim 1, further comprising a plurality of filamentary members extending lengthwise along said tube, said filamentary members reinforcing said tube.

11. A surgical access port according to claim 1, wherein said elastic membrane is thinner in said constricted region than in a region adjacent to said constricted region.

12. A surgical access port according to claim 1, wherein said tube has a plurality of corrugations therein, said corrugations increasing the flexibility of said tube.

13. A self sealing surgical access port permitting a surgical tool to be used within a body cavity, said access port comprising:
a rigid duct having a distal end positionable within the body cavity, said duct being extendable through living tissue of said body and having a proximal end positionable outside of said cavity; and
a flexible tube positioned substantially coaxially within said duct and attached thereto, said tube having an inner low-friction surface surrounded by an elastic membrane, said tube having opposite ends attached to said duct so as to define a pocket between said tube and said duct between said opposite ends of said tube, said pocket being pressurized with a fluid so that said membrane forms a constricted region between said ends of said tube, said tube being elastically deformable radially outwardly against said pressurization to permit said surgical tool to pass through said duct and into said body cavity, said pressurization forcing said constricted region of said membrane to close around said tool to substantially continuously seal said tube while said tool extends therethrough.

14. A surgical access port according to claim 13, wherein said low-friction inner surface is in contact with itself around said tube so as to form a seal substantially closing said duct.

15. A surgical access port according to claim 13, further comprising a valve positioned in said duct for sealing said duct in the absence of a surgical tool inserted therethrough.

16. A surgical access port according to claim 15, wherein said valve comprises a duckbill valve.

17. A surgical access port according to claim 13, wherein said pocket has an annular cross-sectional shape substantially surrounding said tube.

18. A surgical access port according to claim 13, wherein said pocket has a hemi-cylindrical cross-sectional shape.

19. A surgical access port according to claim 13, wherein said tube comprises a laminate formed from a low-friction membrane forming said inner surface, said low-friction membrane being surrounded by said elastic membrane.

20. A surgical access port according to claim 13, wherein said fluid inflating said pocket is a gas.

21. A surgical access port according to claim 13, wherein said fluid inflating said pocket is a liquid.

22. A surgical access port according to claim 13, wherein said fluid inflating said pocket is a gel.

23. A surgical access port according to claim 13, wherein said elastic membrane is biased into a constricted shape so as to augment the biasing of said constricted region of said tube.

24. A surgical access port according to claim 23, wherein said tube is biased into a conical shape.

25. A surgical access port according to claim 23, wherein said tube is biased into an hourglass shape.

26. A surgical access port according to claim 13, further including an insufflation port attached to said duct.

27. A surgical access port according to claim 13, further comprising a vent in fluid communication with a space between said duct and said flexible tube.

28. A surgical access port according to claim 27, wherein said vent comprises at least one aperture extending through said duct.

29. A surgical access port according to claim 27, wherein said vent is in fluid communication with the atmosphere outside said body cavity.

30. A surgical access port according to claim 27, wherein said vent comprises at least one aperture through said flexible tube.

31. A surgical access port according to claim 27, wherein said vent is located distally to said constricted region.

32. A surgical access port, comprising:
a stiff outer shell having a plurality of lengthwise extending struts arranged circumferentially, said struts defining a plurality of slots in spaced relation to one another;
a membrane positioned within said outer shell, said membrane formed from a material having a lower coefficient of friction than said outer shell.
